(19) United States
(12) Patent Application Publication
(10) Pub. No.: US 2010/0268278 A1
(43) Pub. Date: Oct. 21, 2010

(54) TENSION BAND

(75) Inventors: Kevin T. FOLEY, Germantown, TN (US); Regis W. Haid, Atlanta, GA (US); Newton H. Metcalf, JR., Memphis, TN (US); Bradley J. Coates, Yarmouth, IA (US); David Alan Sharp, Oakland, TN (US); Chris Michael Diaz, Memphis, TN (US); Virginia Leigh Richardson, Memphis, TN (US); Jeff R. Justis, Germantown, TN (US); Hai H. Trieu, Cordova, TN (US); Jeffrey H. Nycz, Warsaw, IN (US); Greg C. Marik, Collierville, TN (US); Troy D. Drewry, Memphis, TN (US); Richard J. Thiele, Collierville, TN (US); Gary S. Lindemann, Collierville, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/424,083

(22) Filed: Apr. 15, 2009

(51) Int. Cl. A61B 17/70; A61B 17/84

(52) U.S. Cl. 606/263; 606/264; 606/300

(57) ABSTRACT

Various embodiments of a vertebral tension band assembly and associated connection structure are provided. The tension band assemblies may be attached to vertebral bodies to, for example, connect one vertebrae to another, retain the band in approximately a preferred position by application of tension to the band during insertion and/or limit, impede, inhibit, reduce or interfere with the separation from one vertebra to another and may further block, impede, interfere with, inhibit, reduce or present an obstacle to dislodgement of a spinal implant from between the vertebrae to which it is attached. Flexible band portions of the assemblies may be treated and/or configured to promote bony integration between the band and the associated vertebrae, limit tissue adhesion to the band, and/or to elute a therapeutic substance from the installed band to the surgical site.

### BACKGROUND OF THE INVENTION

The present invention generally relates, for example, to apparatus used to correctively link two or more vertebral bodies, and more particularly relates to tension band apparatus connectable to vertebral bodies.

Various flexible tension band assemblies have been previously proposed for attachment to vertebral bodies to limit the separation therebetween, and to outwardly block dislodgement of an implant disposed between the vertebral bodies. Several problems, limitations and disadvantages have commonly been associated with tension band assemblies of conventional design, including inaccuracies in achieving in-place tensioning thereof, lack of ease and reliability in the securement of the assemblies to the vertebral bodies, and the need to provide associated therapeutic and other treatment to the surgical site of the tension band installation. A need exists for alleviating these problems, limitations and advantages. It is to this need and others that various embodiments of the present invention are directed.

### SUMMARY OF THE INVENTION

In carrying out principles of the present invention, in accordance with various representative embodiments thereof, vertebral linking assemblies, and associated connection structure, are, for example, provided for attachment to vertebral bodies in a manner that may either hold the assembly in a preferred position by the surgeon and/or affect, inhibit, contribute to or limit the separation of the vertebral bodies to which it is attached and/or cover, inhibit, or reduce the likelihood of subsidence and/or blocking dislodgement of a spinal implant from between the vertebrae. Representatively, the linking assemblies may, for example, include elastic, woven, knitted and/or braided fabric tension band embodiments, or combinations thereof, which may be operatively connectable to the vertebral bodies in a manner such that the band may, for example, exteriorly span at least one disc space between the vertebral bodies or span any two bone portions.

Various connection structures may be used to operatively secure the band to the vertebral bodies, and the band may be modified in several manners to increase its usefulness. For example, a visual and/or radiographic indicia may be placed on the band to provide the physician with a visual and/or radiographic assessment of the amount of tension present in the installed band as well as or alternatively, for example, providing information regarding the placement and/or orientation of the band. Additionally, the band may be configured and/or treated in a manner promoting bony integration between the band and the associated vertebrae, limit tissue adhesion to the band, and/or to elute a therapeutic substance from the installed band.

In accordance with one illustrative embodiment of the invention, a device is provided for linking first and second vertebral bodies having an implant therebetween. The device comprises a flexible member having first and second ends and being formed from a flexible and/or elastic material which may be stretched to place the flexible member in tension, the flexible member having a surface on which indicia is disposed that provides a physician with a visual and/or radiographic assessment of the degree of tension in the flexible member and/or orientation and/or location of the flexible member. Connector apparatus may be provided for securing the first and second ends of the flexible member to at least one of the first and second vertebral bodies in a manner causing the flexible member to remain generally in a desired location and/or to for example limit, inhibit, or otherwise affect separation of the first and second vertebral bodies and/or to inhibit, in part cover, or reduce the likelihood of or block dislodgement of the implant from between the vertebrae.

The flexible member may be configured, or a treatment may be added to at least a portion of the flexible member, to promote bony integration between the flexible member and the first and second vertebral bodies to which it is connected, or to reduce post-operative soft tissue adhesion to the flexible member. Further, the flexible member may be treated with a therapeutic substance, with the installed flexible member being operative and/or adapted to elute the therapeutic substance, over time, to the surgical site.

The connector apparatus may comprise, for example, first and second end caps, respectively secured to the first and second ends of the flexible member, having one or more openings formed therein through which one or more fastening members may be extended into the first and second vertebral bodies to operatively secure the device thereto. Alternatively, the connector apparatus may comprise one or more mechanical fasteners extendable through the flexible member into at least one of the first and second vertebral bodies. The flexible member may, alternatively, be glued, adhered, or otherwise stuck or affixed to one or more of the vertebral bodies and/or intervertebral implant(s). Additionally, tunnels may extend into at least one of the first and/or second vertebral bodies, with portions of the flexible member being disposed within the tunnel or tunnels, and the connector apparatus may comprise one or more fastening members, formed from shape memory material and/or having radially expandable structures, positionable in interference fits within the tunnels to bear against one or more of the flexible member portions and captively retain them in the tunnel or tunnels.

As a further alternative, the connector apparatus may comprise one or more connector structures, each being connectable to the first and/or second ends of the flexible member, for securing the first and/or second ends of the flexible member to at least one of the first and second vertebral bodies. Each of the connector structures may, for example, comprise (1) a hollow locking structure with a base wall having an opening therein and being securable to one of the first and/or second ends of the flexible member, and an outer wall spaced apart from and parallel to the base wall, the outer wall having an opening therein which opposes the base wall opening and is partially bounded by one or more resiliently deflectable lobe portions of the outer wall, and (2) a fastening member such as, for example, preferably a screw or otherwise a tack, staple, pin or other fastener, extendable sequentially through the outer wall opening, the base wall opening and the one of the first and/or second ends of the flexible member, and preferably threadable or otherwise insertable into one of the first and/or second vertebral bodies, the fastening member further having a transversely enlarged head portion configured to resiliently deflect the one or more lobe portions inwardly, as the fastening member passes through the interior of the hollow locking structure, and then permit the deflected lobe portion(s) to snap back to its undeflected position in which it may block or otherwise obstruct or inhibit at least in part outward passage of the head portion through the outer wall opening.

In accordance with another illustrative embodiment of the invention, a device is provided for linking first and second vertebral bodies having an implant therebetween, the device comprising a linking member having first and second ends, and a plurality of connector structures, connectable to the first and second ends of the linking member, for securing the first and second ends of the linking member to at least one of the first and second vertebral bodies.

Illustratively, each of the first and second ends of the linking member may have a mounting hole extending therethrough, and the plurality of connector structures comprises one or more of (1) first and/or second fastening members (such as, for example preferably a screw or alternatively a tack, staple, pin or other connector) each having a head portion through which an opening axially inwardly extends, the first and/or second fastening members being respectively threadable into the first and second vertebral bodies, (2) first and/or second elongated guide members, each configured to be respectively and removably inserted into the openings in the first and/or second fastening members, after the fastening members are threaded, inserted, attached, affixed and/or applied into or on their associated vertebral bodies, and to thereafter have longitudinal portions projecting outwardly from the first and second fastening members, the longitudinal portions of the guide members being movable away from or toward one another by exerting a separation or closing force thereon to increase or decrease the separation distance between the first and second vertebral bodies to facilitate the insertion therebetween of a supportive implant that substantially maintains the increased or decreased separation distance when the separation force is removed or applied from or by the outwardly projecting portions of the first and second elongated guide members, and thereby positions the outwardly projecting portions so that they can extend through the mounting holes of the linking member and permit the first and second linking member ends to be moved therealong into adjacency with the fastening member head portions, and (3) first and/or second locking members constructed and operative to respectively lock the first and/or second linking member ends to the head portions of the first and/or second fastening members before or after the first and second elongated guide members are respectively removed from the first and/or second fastening member openings.

In accordance with a further illustrative embodiment of the invention, multilevel apparatus may be provided for linking first and second vertebral bodies having a third vertebral body disposed therebetween, the apparatus comprising (1) a generally band-shaped flexible structure having first and second ends, and an elongated slot extending through a longitudinally intermediate portion of the flexible structure and longitudinally extending parallel to the length of the flexible structure, (2) first connection structure for respectively securing the first and second ends of the flexible structure to the first and second vertebral bodies, and (3) second connection structure, extendable through the slot through a selectively variable longitudinal portion thereof and securable to the third vertebral body, for securing a longitudinally intermediate portion of the flexible structure to the third vertebral body.

The flexible structure may be of a looped configuration, with a first connection structure comprising first and second end plates having slots therein through which the first and second end portions of the flexible structure respectively extend, openings through which fasteners such as, for example, screws, pins, staples, rivets, or other such devices may be extended and, for example, threaded, pressed, inserted or otherwise attached into or on the first and/or second vertebral bodies. A second connection structure may comprise a grommet configured to outwardly overlie the flexible structure at a portion of the slot, and a fastener extendable through the grommet and threadable or otherwise inserted, attached into or attached or applied onto the third vertebral body.

### BRIEF DESCRIPTION OF THE DRAWINGS

FIGS. 1 and 2, respectively, are inner and outer side perspective views of a tension band structure embodying principles of an embodiment of the present invention and representatively including a flexible tension band portion connected at its opposite ends to end cap members which, in turn, are connectable to vertebral bodies.

FIG. 2A is a schematic anterior side elevational view of an embodiment of the tension band structure of FIGS. 1 and 2 operatively installed on and linking first and second vertebral bodies.

FIGS. 3A-4B elevationally depict, in schematic form, two representative embodiments of a flexible tension band member having disposed thereon indicia providing a physician with a visual and/or radiographic assessment of the tension, location, and/or orientation of the band member.

FIGS. 5A-5E schematically depict several representative embodiments of tension band members operatively spanning two adjacent vertebral bodies and secured thereto by a variety of simple mechanical fasteners extending directly through opposite ends of the band members into the vertebral bodies.

FIGS. 6A-14 cross-sectionally depict the attachment of representative embodiments of tension band members to vertebral bodies using various shape memory-capable and radial expansion-capable mechanical fasteners.

FIGS. 15-16C schematically illustrate a technique for fastening various embodiments of a tension band member to a vertebral body utilizing an embodiment of a specially designed flexible petal fastener locking mechanisms, attached to the tension band member, and associated bone fasteners.

FIG. 16D schematically illustrates a schematic view of an anti-rotation structure or locking mechanism that can be used with the present invention.

FIGS. 17-19 schematically illustrate an exemplary technique for fastening various embodiments of a tension band member, or other type of linking member, to two vertebral bodies, using an embodiment of a specially designed caspar pin-based anchoring system.

FIGS. 20 and 20A illustrate two representative embodiments of specially designed end caps used to secure opposite ends of a tension band member to two vertebral bodies.

FIGS. 21-22B schematically illustrate the use of various fabric-based tension band member embodiments in which the weave, knit or braid of the band member is adjusted, or a treatment is added to at least a portion of the band, to add useful effects to the operatively installed band member such as to promote bony growth between the band and the vertebrae, reduce tissue adhesion to the band, or to provide the elution over time of therapeutic substances from the installed band member.

FIG. 23 is an outer side perspective view of a slotted band embodiment of a tension band structure embodying principles of an embodiment of the present invention.

FIG. 24 shows a schematic view of another embodiment of a multilevel tension band assembly.

FIG. 25 schematically illustrates an “add on” band according to the present invention.

FIG. 26 shows an perspective schematic view of a holding device for use in cooperation with the band assembly according to the present invention.

FIG. 27A shows an perspective schematic view of another embodiment of a holding device according to the present invention.

FIG. 27B shows a front schematic view of holder of FIG. 27A in cooperation with a band assembly according to the present invention.

FIG. 28A shows an perspective schematic view of another embodiment of a holding device according to the present invention.

FIG. 28B shows a cut-away schematic view of the embodiment of an end cap of an embodiment of a tension band assembly that works in cooperation with the holding device of FIG. 28A.

FIG. 29 shows a schematic perspective view of a anti-rotation device according to the present invention.

FIG. 29A shows a cut-away, enlarged cross-sectional schematic view of the distal end of the anti-rotation device of FIG. 29.

### DETAILED DESCRIPTION OF VARIOUS EXEMPLARY EMBODIMENTS OF THE PRESENT INVENTION

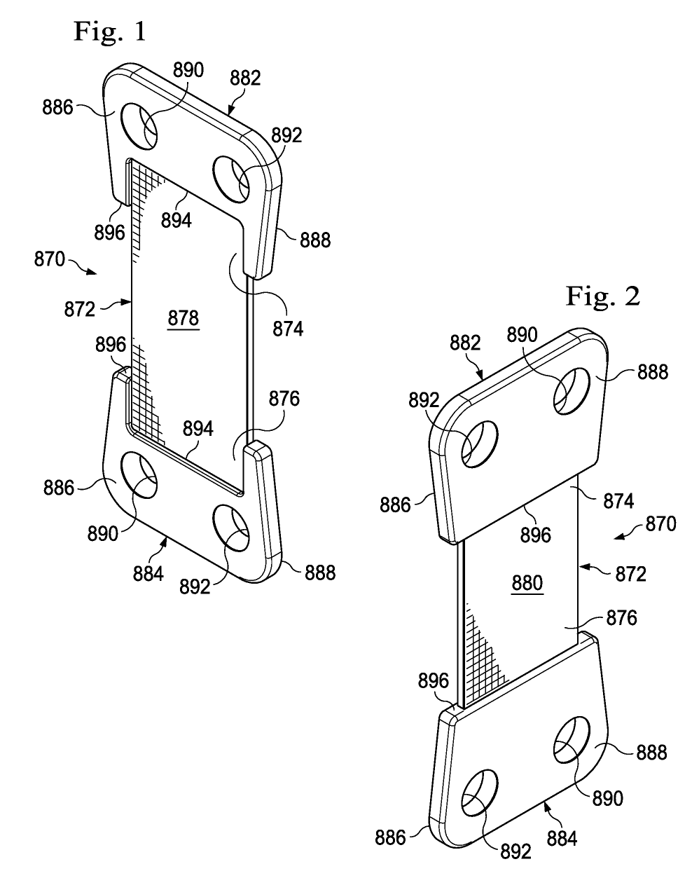

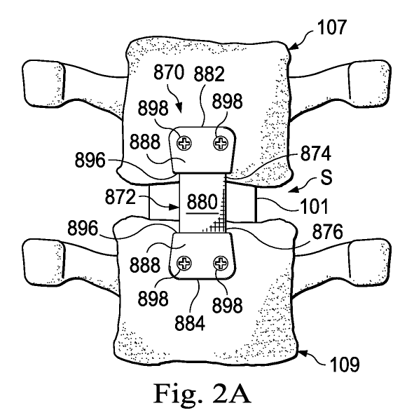

Turning first to FIGS. 1 and 2, perspectively illustrated therein is a vertebral linking apparatus in the form of an assembly 870 which may be utilized, as subsequently described herein, to link two vertebral bodies (such as the upper and lower vertebrae 107,109 in FIG. 2A) in a manner that may, for example (1) assist in holding the apparatus in a preferred location and/or orientation and/or affect, inhibit, contribute to or limit separation of the vertebral bodies, and (2) cover, inhibit or reduce the likelihood of subsidence of or enhance the potential blocking or hinder the dislodgment of a spinal implant (such as the illustrative implant 101 schematically depicted in FIG. 2A) from between the vertebral bodies. Implant 101 may have a variety of different configurations and structures to suit the particular surgical procedure. For example, implant 101 may be a spacer such as a cage for stabilization or an artificial disc for preserving motion.

Vertebral linking assembly 870 comprises a flexible linking member, preferably in the form of a tension band 872 formed from an elastic, woven, knitted or braided or flexible material including but not limited to a cloth, polymer, metal, or tissue or combination thereof, and having first and second ends 874 and 876. Tension band 872 may be formed from a wide variety of suitable materials, including natural or synthetic tissue biocompatible materials. Natural materials include autograft, allograft and xenograft tissues including but not limited to bone and ligaments. Synthetic materials include metallic materials and polymers. The metallic materials can be formed from shape memory alloy, including shape memory materials made from, for example, the nickel-titanium alloy known as Nitinol (“NiTi”). The shape memory materials may exhibit shape memory, but preferably exhibit superelastic behavior. Other metallic materials include titanium alloy, titanium, stainless steel, and cobalt chrome alloy. Suitable polymeric materials include, for example, polyethylene, polyester, polyvinyl, polyvinyl alcohol, polyacrylonitrile, polyamide, polytetrafluoroethylene, poly-paraphenylene, terephthalamide and combinations thereof. Some woven, knitted or braided materials may, for example, include nylon, Dacron®, and/or woven fibers or filaments of polyester, polyethelene, polypropylene, polyetheretherketone (“PEEK”), polytetrafluoroethylene (“PTFE”), woven PEEK, and/or Bionate® or Pursil® manufactured by DMS PTG, Inc. of Berkeley, Calif. Some elastic materials may, for example, include latex, rubber, silicone, polyurethane, silicone-polyurethane copolymers, and/or polyolefin rubbers. Other suitable materials may, for example, include Gore-Tex®, Kevlar®, Spectra, polyether, polycarbonate urethane, shape memory material with pseudo elastic or superelastic characteristics, metals, metal alloys, and polymers, braided polymers, materials made of bone, any bio-compatible material such as an elastomer, demineralized bone, or flexible composite material, ceramic materials, carbon fiber, other natural materials such as allograft, autograft and xenograft, polyacrilonitrile, glass fiber, collagen fiber, ceramic fiber, synthetic resorbable materials such as polyactide, polygycolide, polyorthoester, calcium phosphate, and/or glass, nonresorbable polyethylene, cellulose, materials that are potentially absorbable, and/or materials that are used in making artificial ligaments. In addition to woven, braided, or knitted structures, the band 872 also may be composed of non-woven structures such as non-woven mesh, or chained structures. Tension band 872 has an inner side 878 and an outer side 880.

The assembly 870 further comprises first and second end cap connection members 882, 884 each having inner and outer sides 886, 888, one or more connection holes 890, 892 extending therethrough, and one or more rectangular notches 894 formed in the inner end cap sides 886 and extending inwardly from flat edge portions 896 of the end caps 882, 884. A single hole may be utilized in the end caps 882, 884 to replace the illustrated dual holes 890, 892 therein if desired. End caps 882, 884 may be formed from a variety of alternative materials including, by way of non-limiting example, metal or a plastic material such as PEEK. Additional materials that the end caps 882, 892 could be made of include metals, ceramics and other polymers, and could also include absorbables or adsorbables like Hydrosorb and natural materials like bone, and other tissue—natural or processed—PEK, Polyglycolic Acid, Hydroxyappetite (HA), or a stiffer fabric portion. If the end caps 882, 884 are made of PEEK, they may be flexible and/or elastic to a certain degree. This will add to the flexibility of the overall tension band assembly.

The ends 874, 876 of the tension band 872 may be disposed in the end cap notches 894 and may be secured to the end caps in a number of alternative manners including, by way of non-limiting example, clamping, looping the band material through slots in the end caps and then stitching or welding the band material to itself, welding the band to the end caps, suturing the band to the end caps, and gluing or otherwise adhesively securing the band to the end caps. Welding methods may include ultrasonic or regular heat welding, and other methods may include molding, pressurizing, and stapling.

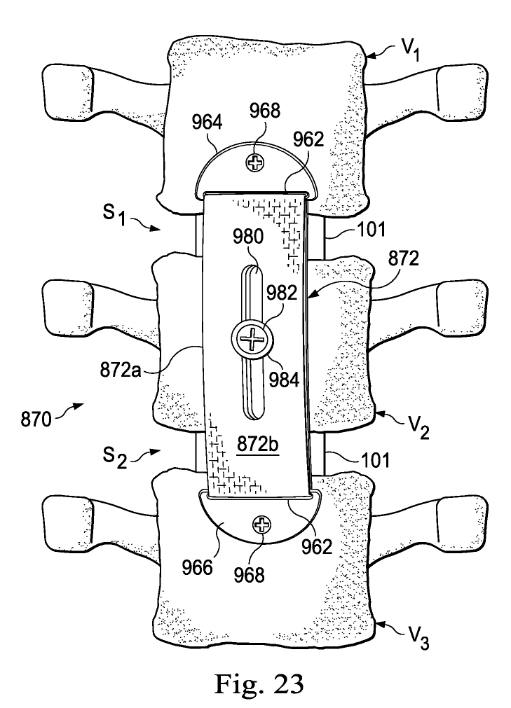

FIG. 2A schematically depicts an embodiment of a tension band assembly 872 operatively secured to the vertebral bodies 107,109 by respectively anchoring the end caps 882, 884 to the vertebral bodies 107,109 using fasteners 898 (such as screws, tacks, pines or staples) extended through the end cap connection holes 890, 892 (see FIGS. 1 and 2) and threaded, inserted, pushed, advanced and/or affixed into the vertebral bodies 107,109. In the installed assembly 870, the band 872 is preferably in tension, thereby resiliently limiting, inhibiting, adjusting or affecting the separation of the vertebral bodies 107,109 from one another or merely connecting thereto and preferably retaining the band in approximately a preferred location and/or orientation, and externally spans the intervertebral space S between the vertebrae 107,109 to thereby block, inhibit, obstruct, or reduce the potential for dislodgement of or otherwise cover at least in part the implant 101 from between them. As illustrated in this embodiment, there may be no direct connection between the tension band assembly 870 and the implant 101. Such direct connection could, however, be utilized if necessary or desirable. Examples of techniques for attaching an intervertebral implant/artificial disc to the band representatively include gluing, heating, and fastening by tack, screw, staple, etc. Additionally, a wrap around with a built-in snap fastener could be used to operatively connect the band to the implant. Alternatively, the implant may be integrally formed or manufactured with and/or as part of the band. Although the depicted tension band illustratively spans a single intervertebral space S, it will be readily appreciated by those of skill in this particular art that it could be easily modified to span a plurality of intervertebral spaces. For example, an extra end cap and length of tension band material could be joined to the illustrated assembly 870, with the three end caps being secured to three vertebral bodies in a manner causing each of the two band lengths to externally span a different one of two intervertebral spaces. Alternatively, an embodiment with one, two or no end caps could be employed to span any particular space or combination of spaces attached by any of the described manners to various vertebra, bones, and/or implants. Other techniques for spanning a plurality of intervertebral spaces (for example, as shown in the subsequently discussed FIG. 23 herein) could also be utilized.

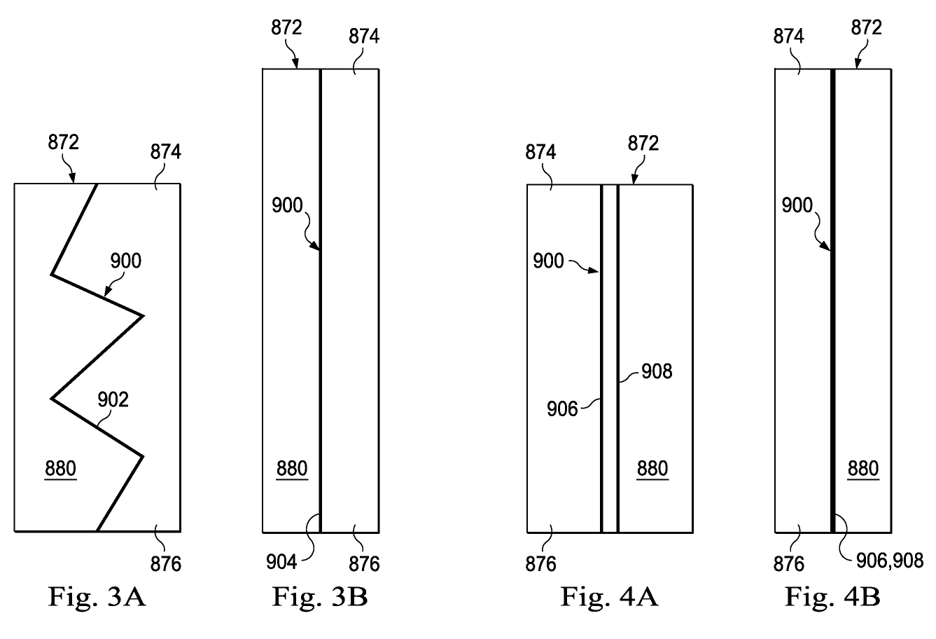

Turning now to FIGS. 3A and 3B, an alternate embodiment of a flexible tension band 872, such as, for example, formed of a fabric and/or elastic material, may have formed on a side thereof (illustratively its outer side 880) visual and/or radiographic tension, location and/or orientation indicia designed to provide a surgeon with a visual and/or radiographic assessment of the tension, location and/or orientation to which the band 872 is subjected after operative connection of the band to its associated vertebrae. For example, with the band 872 is its relaxed orientation as shown in FIG. 3A, the indicia 900 may be configured to assume the indicated zig-zag line configuration extending along the length of the band 872. However, when the band 872 is operatively tensioned in a longitudinal direction to a predetermined degree (as shown in FIG. 3B), the zig-zag line 902 may be deformed to a straight line configuration 904. The indicated indicia 900 may provide the surgeon with both a visual assessment (before the incision is closed) of the band tension, and a radiographic assessment (after the incision is closed) of the band tension, by simply forming the indicia from a suitable radio-opaque material of a color, contrast, density, or other image that contrasts with the color, contrast, density, or other image of the band 872.

FIGS. 4A and 4B depict one of many possible alternatives to the shape of the indicia 900 shown in FIGS. 3A and 3B. With the band 872 in its relaxed orientation (FIG. 4A) the modified indicia 900 may be shaped as two parallel, laterally spaced apart single straight lines 906 and 908. When the band 872 is tensioned to a predetermined degree (as in FIG. 4B), the lines 906, 908 may laterally merge to form a single, thicker line 908. As can be seen in FIGS. 3B and 4B, the indicia lines 904 and 906,908 therein each may provide the surgeon with a useful centerline marker which may be used to verify correct lateral centering alignment between the band and the patient's spine.

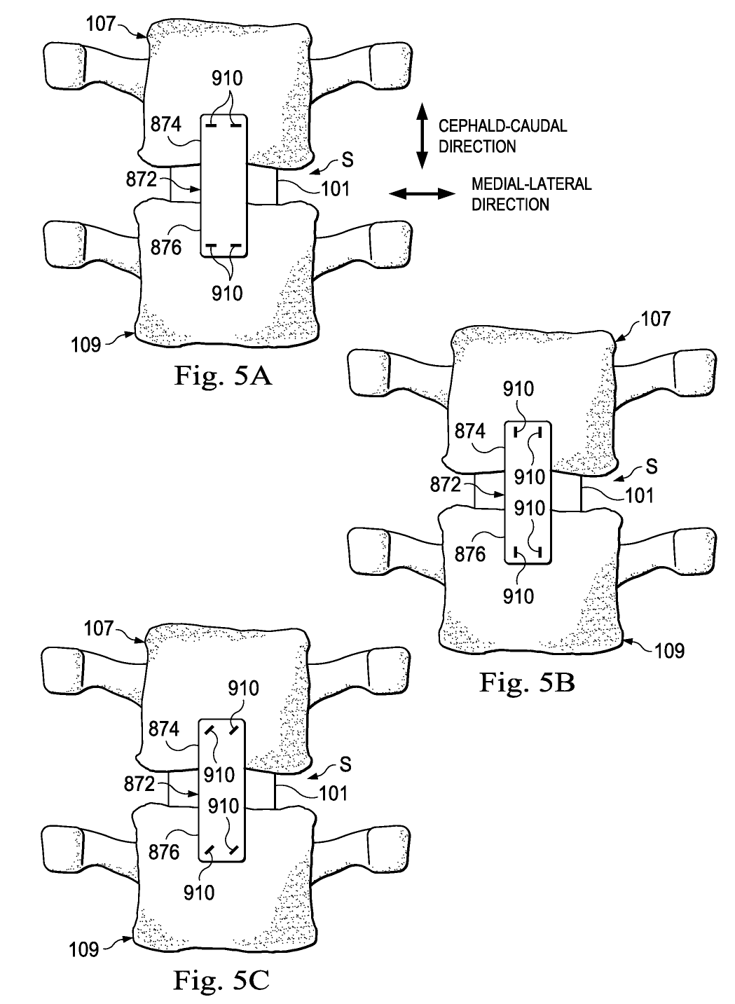

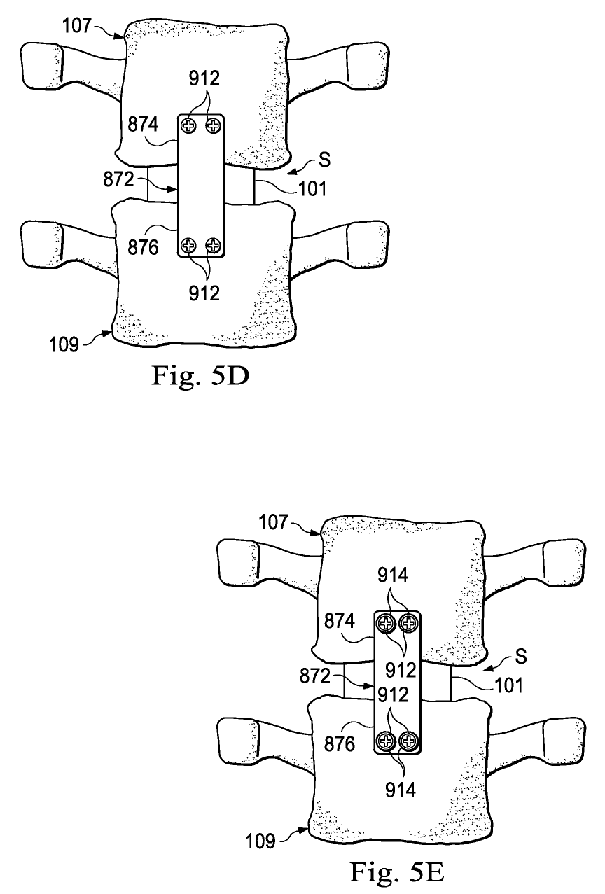

FIGS. 5A-14 illustrate various techniques for directly securing vertebral linking members, such as one or more of the previously described tension band members 872, to first and second vertebral bodies, without using the previously described end cap members 882 and 884, using a variety of simple mechanical fastening members extended through or forcibly contacting the vertebral linking members.

For example, as shown in FIGS. 5A-5C, one or more staples 910 may be driven directly through the band ends 874,876 into the vertebral bodies 107,109 to operatively secure the tension band 872 to the vertebral bodies 107,109 in a manner such that the band 872 is, to some degree, held in a desired position and/or resiliently resists, inhibits and/or reduces further separation of the vertebral bodies from one another, and externally spans the intervertebral disc space S in a manner blocking at least in part, limiting, inhibiting or reducing the likelihood of dislodgement of the implant from space S and/or at least in part covers the implant or is in part attached to the implant. As illustrated, the lengths of the one or more staple bases may extend in a medial-lateral direction (FIG. 5A), a cephald-caudal direction (FIG. 5B), or in any direction between these two directions (FIG. 5C). The staples 910 may be rigid, or their bases may be at least somewhat flexible, in order to control the degree of band retention forces provided by the installed staples.

As shown in FIG. 5D, bone screws 912 may be extended directly through the opposite ends 854, 876 of the tension band 872 and threaded into the vertebral bodies 107 and 109, or, as shown in FIG. 5E, the bone screws 912 may be extended through grommeted openings 914, pre-formed in the band ends 874 and 876, and threaded into the vertebral bodies 107,109. The grommets 914 may be of a rigid material such as metal or plastic, or simply be stitched cloth hole reinforcing grommets. Alternatively, instead of grommets, the band may include formed, heated, cut, welded, reinforced, and/or otherwise created openings in the material for receiving a fastener or attaching compound or material of some shape or form.

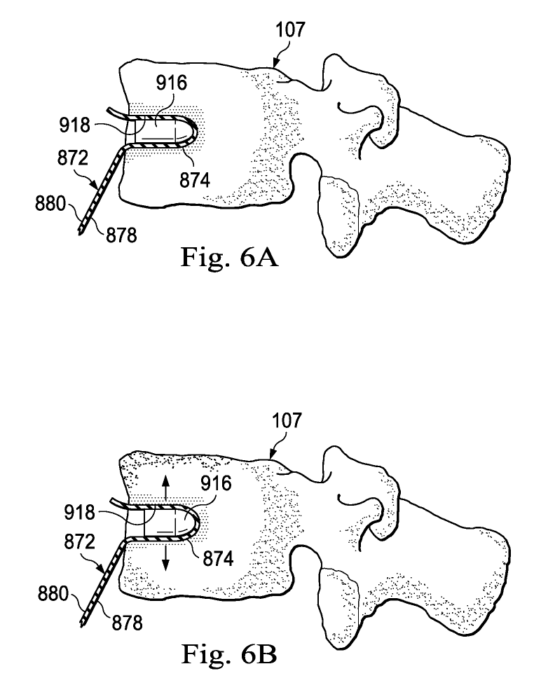

Other techniques for utilizing mechanical fasteners to directly connect the tension band 872 (or other type of flexible vertebral linking member as the case may be) to vertebral bodies are shown in FIGS. 6A-14 and use what may be generally termed interference type fasteners—representatively either temperature-activated shape memory fasteners or radially expandable type mechanical fasteners.

Cross-sectionally illustrated in schematic form in FIGS. 6A and 6B is a shape-memory type interference pin 916 used to anchor an end 874 of a flexible vertebral linking member 870 (such as the previously described elastic tension band) to one of the vertebral bodies 107. To achieve this anchoring, a tunnel 918 is first formed in the vertebral body 107, and the linking member end 874 is suitably inserted into the tunnel 918. Then, with the interference pin 916 at a temperature less than the patient's body temperature (and thus in a radially compressed configuration) the pin 916 is inserted into the tunnel 918, so that the pin 916 is within the looped band end 844 (see FIG. 6A), and allowed to warm up to the patient's body temperature, thereby radially jamming the looped band end 874 within the tunnel 918 as shown in FIG. 6B.

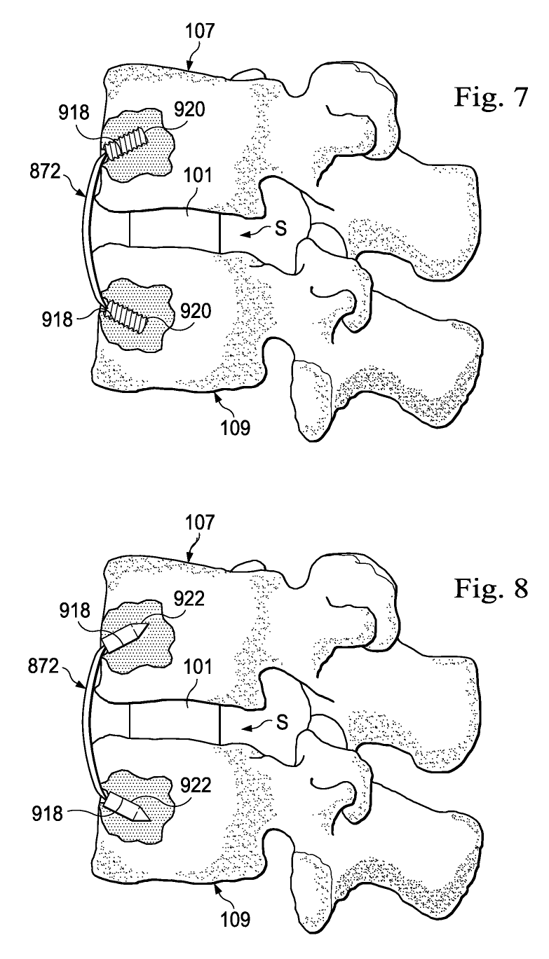

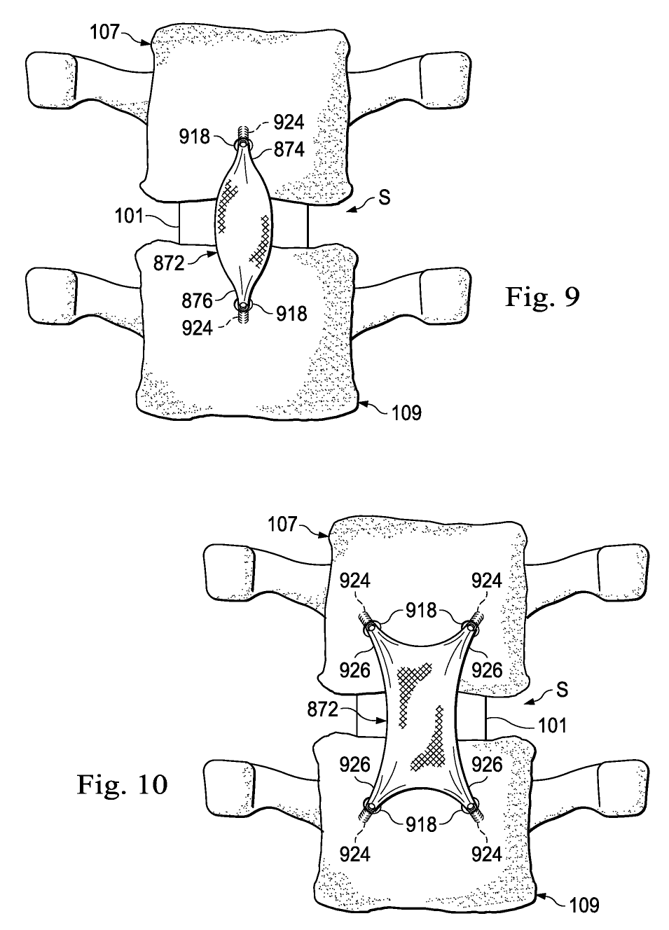

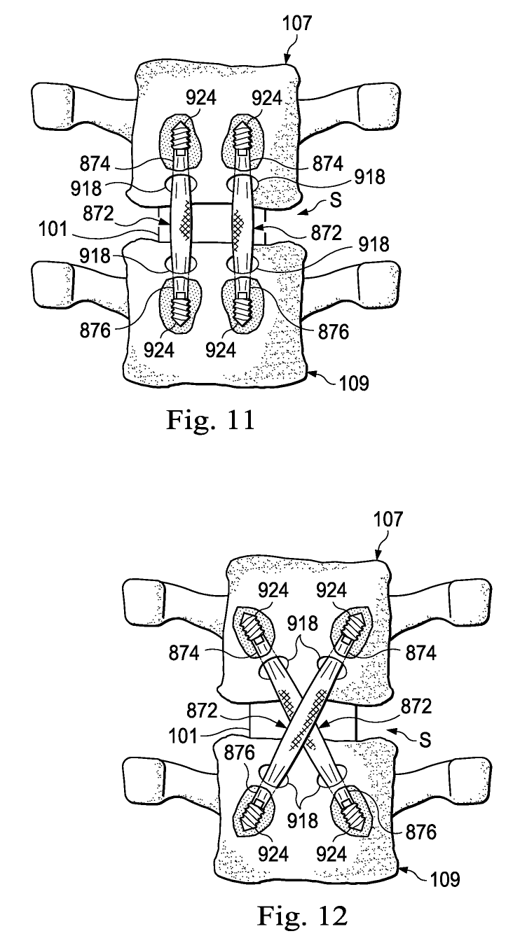

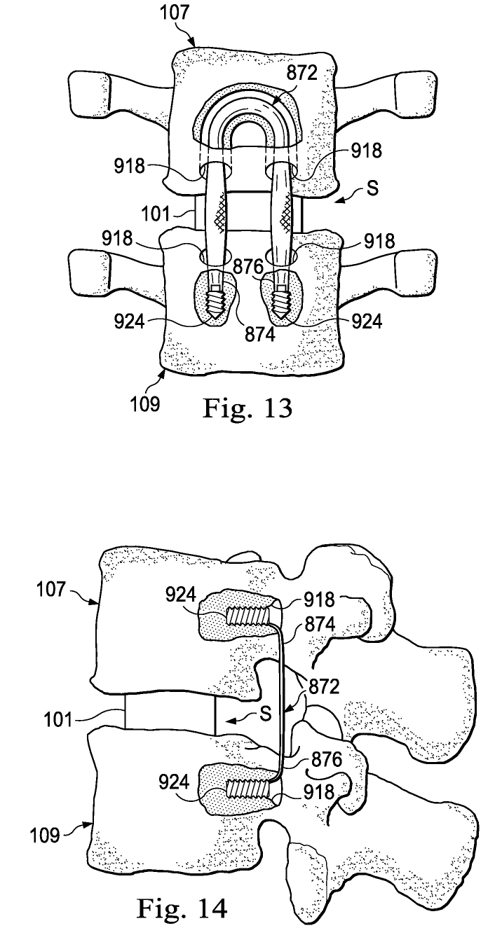

FIGS. 7-14 depict various representative permutations of this interference fastener-based linking member-to-verterbra attachment technique. For example, in FIG. 7 opposite ends of a flexible vertebral linking member 872 are positioned within tunnels 918 formed within the vertebral bodies 107,109 and secured therein using threaded interference screws 920 of a shape-memory type, with the implant 101 between the vertebral bodies 107,109 being a fusion device implant. In FIG. 8, fasteners 922 of the non-threaded shape memory type or mechanical fasteners with mechanical radial expansion capabilities such as, for example, gull anchors, are used to anchor the opposite ends of the linking member 872 in the vertebral tunnels 918. Representatively, the implant 101 in this instance is an artificial disc. In FIG. 9 the flexible linking member 872 is generally band shaped, and has narrowed end portions 874,876 anchored in vertebral tunnels 918 by interference type fasteners 924. In FIG. 10 the flexible linking member 872 is somewhat wider, and has corner end portions 926 thereof anchored in vertebral tunnels 918 by interference type fasteners 924 as previously described. FIGS. 11 and 12 respectively show parallel and crossed pairs of flexible vertebral linking members 876 secured to associated vertebral bodies using interference type fasteners 924. FIG. 13 illustrates a flexible vertebral linking member 872 which is looped through a generally U-shaped tunnel 918 formed in the upper vertebral body 107, and has its ends 874,876 secured within a pair of tunnels 918 in the lower vertebral body 109 by interference type fasteners 924. FIG. 14 shows a flexible vertebral linking member 872 secured to a posterior side of vertebral bodies 107,109 using interference type fasteners 924.

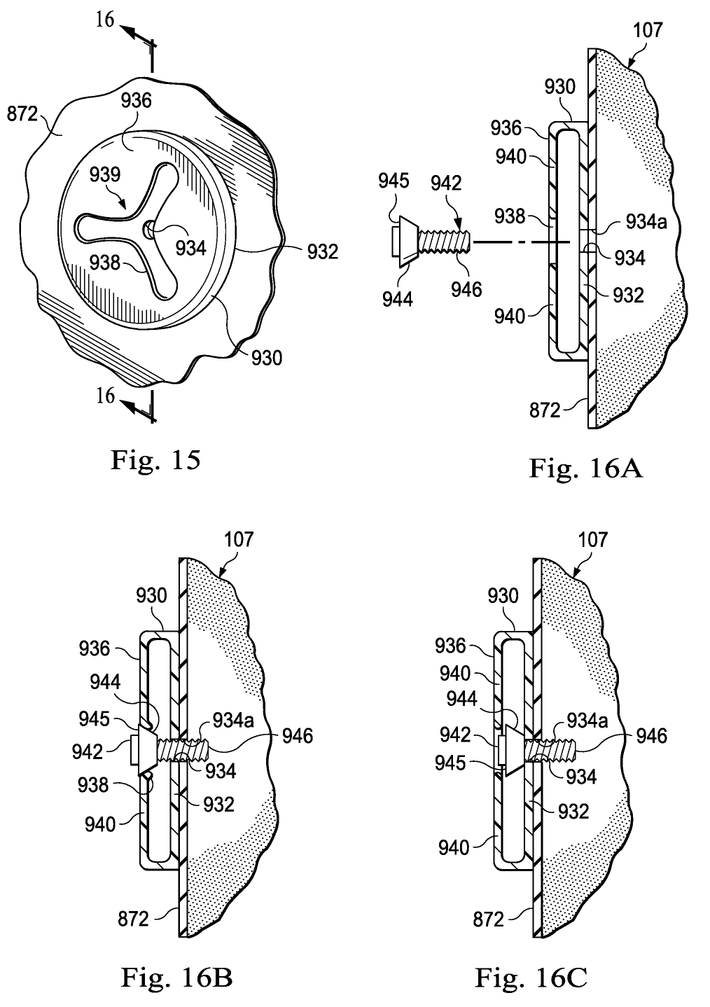

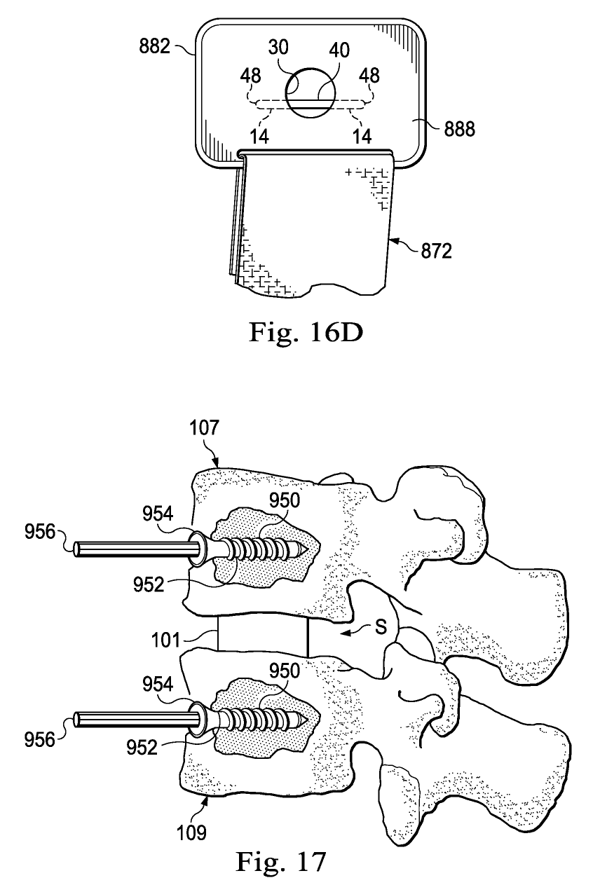

FIGS. 15-16C illustrate a specially designed mechanical fastening structure for use in attaching the opposite ends 874,876 of the tension band 872 (or another type of vertebral linking member as the case may be) to the vertebral bodies 107,109. In an exemplary form thereof, such structure includes a hollow, generally cylindrical locking body 930 having a circular base wall 932 with a central mounting hole 934 extending therethrough, and a spaced apart, facing circular outer wall 936 with a generally petal-shaped central opening 938 that overlies the mounting hole 934 and has a circumferentially spaced one or more inwardly projecting, resiliently deflectable lobe portions 939. To ready the locking body 930 for use, its base wall 932 is suitable secured to the outer side of the flexible tension band 872, as by a sonic welding or other securement process, so that the central mounting hole 934 overlies a corresponding mounting hole 934a formed in the tension band 872.

The fastening structure may also comprise, for example, a bone screw 942 having a tapered head portion 944 on which an annular outer end ledge 945 is defined. To use the fastening structure to, for example, secure a tension band end to the vertebral body 107, the tension band end is placed over the desired mounting location on the vertebral body 107 (see FIG. 16A), and the threaded body portion 946 of the bone screw 942 is sequentially extended through the locking structure openings 938, 934, and the linking member opening 934a, and then threaded into the vertebral body 107.

As the tapered screw head 944 passes into the interior of the locking body 930, it inwardly deflects the one or more lobes 940 (see FIG. 16B), and then inwardly passes them, permitting them to snap back to their undeflected positions in which they overlie the annular screw head ledge 945, thereby forming a barrier relative to this portion of the screw which blocks the screw 942 from backing out of the vertebral body 107. It is to be understood, of course, that a plurality of fastening structures as just described can be used at each end of the linking member to be attached to the representatively illustrated vertebral bodies 107,109.

A variety of anti-rotation structures may be utilized at the screw-cap interface, such as an elevated ramp or a flexible finger, to prevent loosening rotation of the installed screw 942 which could cause it to back out and lift the entire end cap off. This anti-rotation structure could also comprise one or more interfering structures on the cap and the screw head, plate, cap or a wire extending across at least a portion of the opening 938 and/or fastener or attachment compound or mechanism and functioning to retain the screw (or other fastener) head 944 in place.

Certain types of anti-rotation structures or anti-backing-out locking members are disclosed in copending U.S. application Ser. No. 11/863,969, which is hereby incorporated herein by reference in its entirety. FIG. 16D shows a schematic view of an anti-rotation structure 40 that can be used with the present invention. Specifically, FIG. 16D shows an embodiment of an end cap 882 with one connection hole 30 for attachment to a vertebra. The anti-rotation structure or locking member 40 may be press fit into guide holes 14. Note that the size of the guide hole 14 may be slightly smaller than the locking member 40. This sizing allows for the locking member 40 to be forced into the guide hole 14 and form a secure attachment. In addition, the ends 48 of the locking member 40 may protrude out of the guide holes 14 so long as the locking member 40 is securely attached to the end cap 882. In the embodiment shown in FIG. 16D, once the locking member 40 is placed within the guide holes 14, the length of the locking member 40 prevents inadvertent removal of the locking member 40 and forms a secure attachment. Locking member 40 also may be attached to the end cap 882 by another arrangement of guide holes 40, a single guide hole 40, or for example, by a another fastener or adhesive.

Once the end cap 882 is positioned, a mechanical fastener such as a screw (not shown in FIG. 16D) is inserted into the connection hole 30. During insertion, the locking member 40 can yield in a direction away from a center of the connection hole 30 to allow for insertion of the mechanical fastener. After a head of the mechanical fastener passes beyond the locking member 40, the resilient locking member 40 rebounds towards its original position, for example, as shown in FIG. 16D. The locking member 40 extends over the head of the mechanical fastener, thus preventing the mechanical fastener from backing out of the bone and away from the end cap 882. In one embodiment, the section of the locking member 40 that extends across the connection hole 30 is substantially straight prior to insertion of the mechanical fastener and after the fastener moves beyond the locking member 40, whereas in another embodiment, the locking member 40 may have a curved shape to accommodate the geometries of the end cap 882 or other needs. The locking member 40 may be made of resilient material such as NiTi. An added benefit of utilizing such a locking member 40 is that it can be used as a visual aid, for example, to let the surgeon know that the screw is in far enough. When such a locking member 40 is made of NiTi or other radiopaque material, is also can be used as a visual aid for location of the band assembly 870.

Other types of fasteners used to operatively connect a linking member between two vertebral bodies may include hybrid screws as illustrated and described in copending U.S. application Ser. No. 12/423,951, which is hereby incorporated herein by reference in its entirety. These screws are potentially hybrid screws that may, for example, include a sharp extended or elongated tip and that may, for example, be more easily tapped or tacked in and then may be screwed in place. Other types of fasteners could include pins, tacks, staples, or staples with hook ends or other features to keep the staple in the bone or enhance the retention of the fastener in the bone to which it is attached. These fasteners could be resorbable or absorbable and could be made of bone, tissue, plastic, hydrosorb, or a suitable metal material and may be coated with an adhesive, cement, rHBMP, or other material to assist and/or enhance retention of the fastener in or on the bone.

Shown in FIGS. 17-19 is a caspar pin-based system for operatively connecting a linking member between the two representatively illustrative vertebral bodies 107 and 109. With initial reference to FIG. 17, the system includes a pair of hollow bone screw members 950, each having a passage 952 extending longitudinally inwardly through its head portion 954. The system also includes a pair of elongated guide members 956, representatively, for example, caspar posts. To ready the vertebral bodies 107,109 for operative insertion therebetween of the implant 101 (illustratively a bone graft implant), the screws 950 are threaded into the vertebral bodies 107 and 109 as shown in FIG. 17, and the guide members 956 are removably inserted into the screw passages 952. With the guide members 956 operatively inserted into the screw interiors, upper longitudinal portions of the guide members 956 extend upwardly beyond the screw heads 954.

Next, a conventional ratcheting device (of the type used in caspar post procedures and not illustrated herein) may be used to transversely force the removable guide members 956 away from one another to thereby increase the separation distance between the vertebral bodies 107,109 to an extent permitting the implant 101 to operatively be inserted between the separated vertebral bodies 107,109. The ratcheting device is then removed, with the inserted implant 101 now holding the vertebral bodies in their separated orientation shown in FIG. 17.

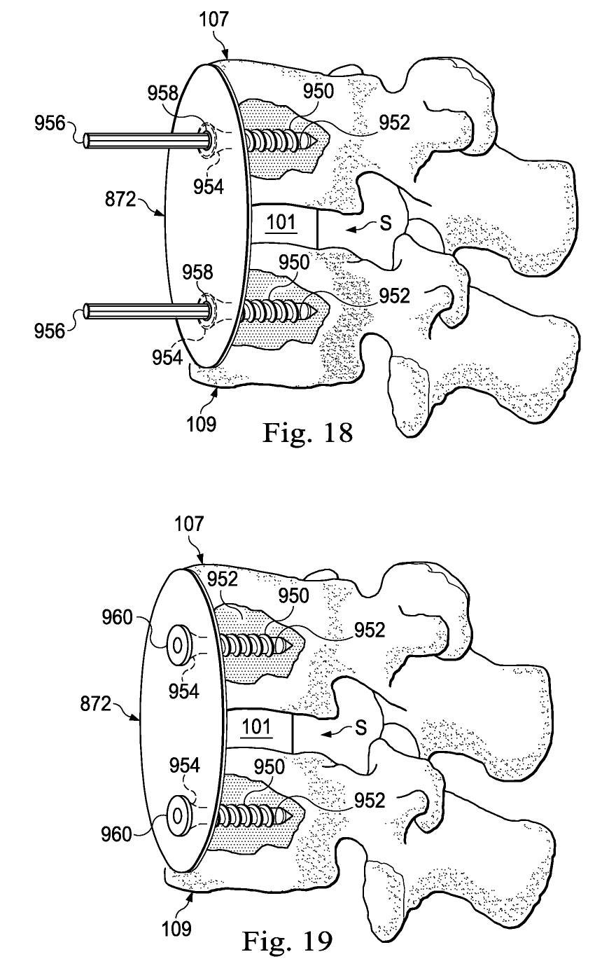

Next, as shown in FIG. 18, the upper ends of the guide members 956 are extended through end holes 958 in the vertebral linking member 872 which, in this exemplary embodiment of the present invention, is a rigid anterior cervical plate but could alternatively be a flexible linking member such as the flexible (which may, for example be elastic and/or woven fabric and/or tissue, metal or some combination thereof) tension band member previously described herein. The linking member 872 is then slid downwardly along the guide members 956 until the linking member's ends reach the screw heads 954. Then, as depicted in FIG. 19, the guide members 956 are pulled out of the hollow bone screws 950 (which are left in place in the vertebral bodies 107,109). Finally, the linking member 872 is anchored in place by securing suitable locking caps 960 to the screw heads 954. Alternatively, a mechanism may be provided which removes the guide members 956 and installs the locking caps 960 simultaneously. Examples of types of mechanisms which may be employed to perform variations of these techniques or steps such as, for example, inserting, guiding and/or removing or moving various elements and/or implants may be found in U.S. Pat. Nos. 7,008,422 and 6,235,028, each of which are hereby incorporated herein by reference in their entirety.

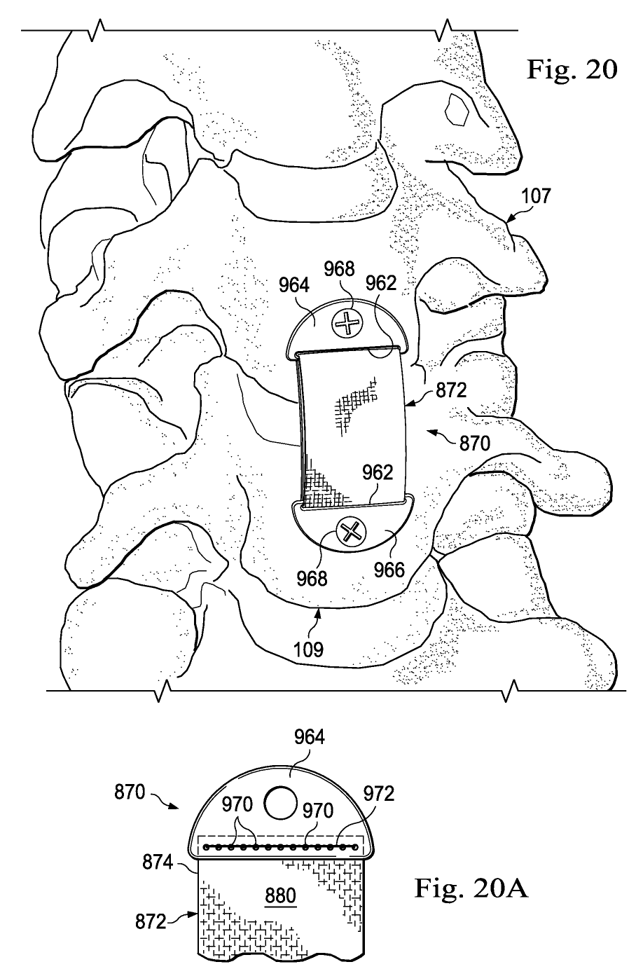

FIG. 20 illustrates an alternate embodiment of the previously described vertebral linking assembly 870 in which an embodiment of a tension band 872 is in a looped configuration, passing through slots 962 in end caps 964, 966 securable to the representative vertebral bodies 107, 109 using bone screws 968 ( or other suitable fasteners or other attaching, adhering or connecting techniques and/or combination of elements) extending through mounting holes in the end caps 964, 966 and threaded into the vertebral bodies 107,109. Compared to passing discrete mechanical fasteners through the tension band 872, the use of these slots greatly reduces undesirable stress concentrations on the tension band 872. Another benefit of using the looped design and slots 962 of FIG. 20 is that it can approximately double the flexibility of the band 872 over the same distance, i.e., length. Advantageously, however, it is helpful for the overall effectiveness and implantation of such a design of the band assembly 870 if each slot 962 is approximately the same width or as close as possible as the width of the band 872. Another technique for reducing such stress concentrations is shown in FIG. 20A in which the end caps 964, 966 (only end cap 964 being shown in FIG. 20A) are each provided with a spaced series of stitch holes 970 therein through which a stitch line 972 may be extended into an underlying end (for example, end 874) of the tension band 872.

According to yet another aspect of an embodiment of the present invention, the exemplary woven, knitted or braided cloth or polymer and/or elastic and/or flexible tension band embodiment of the vertebral linking member 872 may be constructed and/or treated to provide the installed linking member with beneficial post-operative attributes.

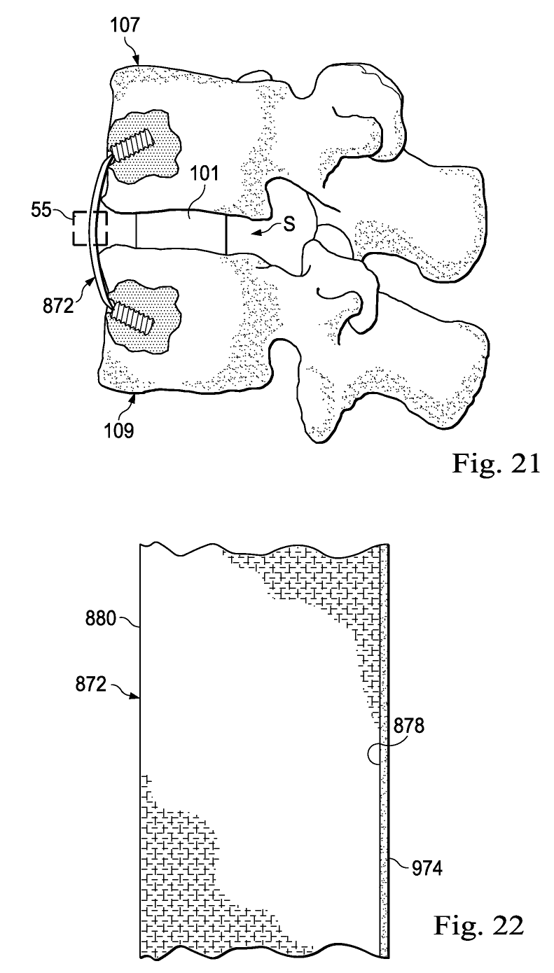

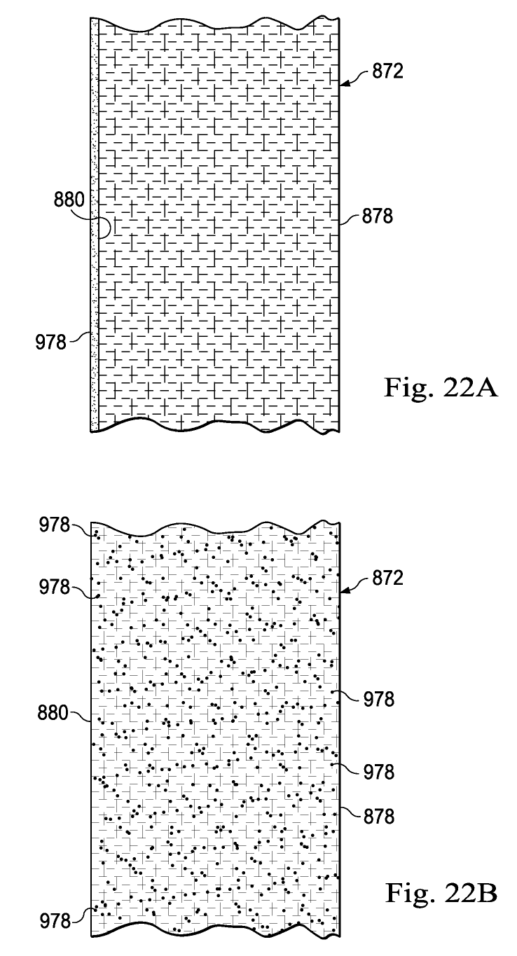

For example, with initial reference to FIGS. 21 and 22, the weave of a woven embodiment of band 872 could be adjusted (for example, to a more porous weave), or a treatment 974 could be added to the inner side 878 of the band 872, to promote bony incorporation into the band from the vertebral bodies 107,109. Examples of such surface treatment may include Hydroxyappetite (“HA”) substances, allografts, biologics, etc. The treatment could potentially expedite healing and add stability to the construct. Additionally, as shown in FIG. 22A, the weave of the band 872 could be adjusted, or a treatment 978 could be added to the outer side 880 of the band 872, to reduce tissue adhesion to the outer side of the band. Such surface treatment or weave adjustment could include known adhesion prevention geometries, barriers, or biologic or non-biologic additives. This feature could potentially reduce the complications associated with soft tissue adhesions such as dyspahgia, vascular adhesions complicating lumbar revisions, etc. Treatments 974,978 could, for example, be rHBMP of various varieties, e.g., BMP2 or BMP 11 or 13—artificial ligaments and/or bone proteins and growth factors. Sponges, bladders, pockets, matrices, substrates, or other materials or capsules or compositions (such as a combination of calcium phosphate and Hyaluronic acid) could be incorporated into the weave to help hold, receive, and/or release the surface treatment. Steroidal treatments or anti-steroidals, antibiotics, pain relievers, medicament, anesthetic, muscle relaxants, tumor necrosis factors, anti-inflammatory, adhesion retardant and hypoallergy treatments may also be utilized.

Finally, as depicted in FIG. 22B, the band 872 which may, for example, be made of a fabric, could be formed from a material adapted to be soaked in a therapeutic substance 978 and then allow for a controlled elution of the substance over time. For example, the eluted substance could be antibacterial, steroidal, anti-inflammatory, pain medication or an anti-scarring substance. The use of this therapeutic elution technique could potentially reduce the incidence of infection and tissue swelling. The slow release of a drug from the band material may be effected using a polymer, embedded capsule, or a wafer. Pockets or bladders can be affixed or sewn on the band material. Further, this drug-eluting technique may comprise both absorbing or adsorbing characteristics. Examples of drugs that may be utilized include Ancef, Vancomycen, and various pain medicines. One specific example is 40 mg. of Depromedrol.

U.S. Pat. No. 7,055,237, which is hereby incorporated herein by reference in its entirety, provides information on how drug eluting capabilities can be applied. For example, a coating (not shown) on the fabric band 872 can be used for a number of purposes, including, but not limited to, a diffusion barrier to control the elution rate of a therapeutic agent from the band 872.

The band 872 may comprise one or more therapeutic agents dispersed within or encased by a polymeric coating (not shown), which are eluted from band 872 with controlled time delivery after installation of the band 872 within a body. As described, a therapeutic agent is capable of producing a beneficial effect against any number of adverse conditions, e.g., inflammation or pain. The elution rates of the therapeutic agents into the body and the tissue surrounding the band 872 are based on the constituency and thickness of how much coating is deposited or incorporated into the band 872, the nature and concentration of the therapeutic agents, the thickness and composition of the particular agent, and other factors.

The coating used to help or control the elution of the agent can be a polymer including, but not limited to, urethane, polyester, epoxy, polycaprolactone (PCL), polymethylmethacrylate (PMMA), PEVA, PBMA, PHEMA, PEVAc, PVAc, Poly N-Vinyl pyrrolidone, Poly (ethylene-vinyl alcohol), combinations of the above, and the like. Suitable solvents that can be used to form a liquid coating include, but are not limited to, acetone, ethyl acetate, tetrahydrofuran (THF), chloroform, N-methylpyrrolidone (NMP), combinations of the above, and the like.

A coating and the above examples are merely exemplary, and it should be recognized that coating configurations other than a basic coating, such as multiple coating layers, are possible. In addition, the coating may cover a portion of the band 872 or the whole band 872.

The different coatings can be made of the same material or different materials, and can contain the same therapeutic agents or different therapeutic agents. Coatings can be applied as a liquid polymer/solvent matrix. A liquid coating can be applied to the band 872 by pad printing, inkjet printing, rolling, painting, spraying, micro-spraying, dipping, wiping, electrostatic deposition, vapor deposition, epitaxial growth, combinations thereof, and other methods as will be appreciated by those skilled in the art.

Initial tensioning of a band member may be achieved using a suitable instrument which may attach to the end caps and could, for example, stretch the band member to a certain or desired tension. Such instrument could include, for example, a reverse pliers (to enable squeezing a handle to expand the ends that might attach to or grip the ends of the band) or combination pliers, retractor, and /or distractor or, for example include an instrument with moving tubes or guide rods such as disclosed in U.S. Pat. No. 7,008,422. The band member may also be tensioned by hand, with the surgeon tacking one end into place and then longitudinally stretching the band member to achieve proper tension before securing its other end to the patient. The band member may illustratively be 14 mm wide to achieve graft containment, and have a suitable length to accommodate patient anatomy. The material of the band member may illustratively be a polyester knit which stretches to that the original band member length increases 30% at 30 Newtons, and reaches failure at about 127 lbs. (approximately 58 Newtons) or at 78% stretch, i.e., when it has increased in length 78%.

FIG. 23 illustrates an embodiment of a “multilevel” previously described vertebral linking assembly 870 in which the tension band 872 is in a looped configuration, passing through slots 962 in end caps 964, 966 securable to the representative vertebral bodies V₁ and V₃, between which vertebral body V₂ is disposed, using bone screws 968 extending through mounting holes in the end caps 964,966 and threaded into the vertebral bodies V₁ and V₃. As can be seen, the tension band 872, which may be represented by an elastically deformable fabric construction, longitudinally spans the vertebral body V₂, as well as spanning a plurality of intervertebral spaces, namely the intervertebral space S₁ disposed between the vertebral bodies V₁ and V₂, and the intervertebral space S₂ disposed between the vertebral bodies V₂ and V₃. Although the band 872 is illustratively depicted as having a looped configuration, it could alternatively be of a non-looped, single layer construction. Additionally, it could be connected to the end caps 964,966 by a variety of different manners previously described herein.

The illustratively looped tension band 872 may have opposing inner and outer side layers 872a and 872b through vertically intermediate portions of which aligned vertically extending slots 962 are formed, such slots combinatively defining a vertically elongated slot 980 extending through the tension band 872. A vertically intermediate portion of the tension band 872 may be secured to the vertebral body V₂ by means of a bone screw 982 threaded into the vertebral body V₂ or by other fasteners such as a tack, pin, staple, or other attaching structures such as a suture, preformed seal, adhesive, mold, or other connection feature or combination of features. Bone screw 982 extends through a grommet 984 that overlies the outer band layer 872b, thereby clamping a vertically intermediate portion of the band 872 to the vertebral body V₂. The presence of the slot 980 facilitates the attachment of the band 872 to the three vertebral bodies by permitting the bone screw 982 and the associated grommet 984 to be easily shifted in a vertical direction relative to the band (already secured at its opposite ends to the vertebral bodies V₁ and V₂), before being secured to the vertebral body V₂, thereby compensating for differences in the heights of the intervertebral spaces S₁ and S₂.

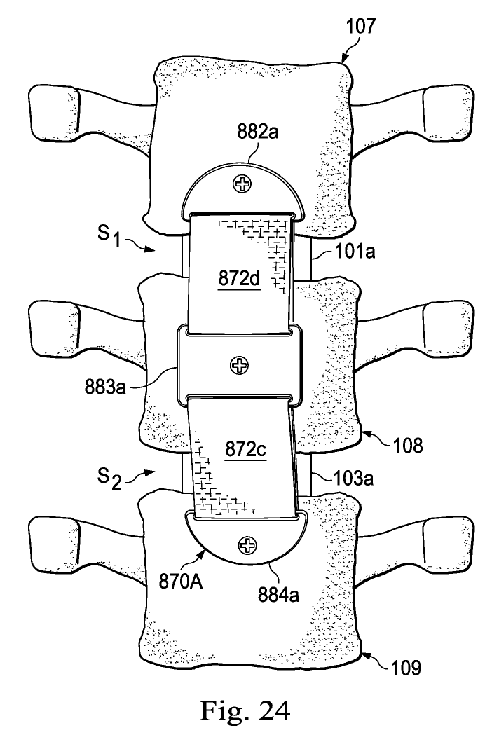

For multiple, consecutive levels of vertebrae as described in FIG. 23, other embodiments may not need the slot 980. For example, one may connect multiple tension bands 872 in series, or end to end, to cover more than one level of vertebrae. A schematic view of an example of such embodiment is provided in FIG. 24, where a multilevel tension band assembly 870A, which here covers two levels, spanning vertebrae 107, 108 and 109, comprises three end caps 882A, 883A, 884A and two bands 872C, 872D. Also, the tension band assembly 870A shown in FIG. 24 contains implants 101A and 103A. The looped version of band 872 may still be utilized in the embodiment of FIG. 24, e.g., with looped bands 872C, 872D.

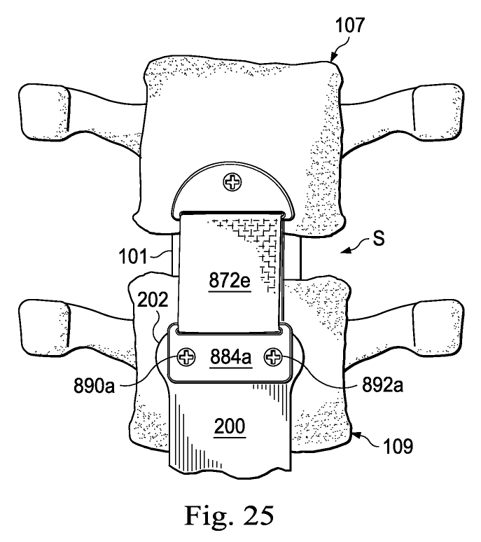

In addition to that described above, patients that have a single or multiple level plate for fixation of vertebrae, for example, can have a tension band 872E added to one end of the existing plate. FIG. 25 schematically illustrates such an embodiment. One benefit of this, as opposed to removing the existing plate 200 and “starting from scratch” is, for example, the ability to address a problem in the spine, inferior or superior to the existing plate on an adjacent level. On such an “add-on” band 872E, another beneficial feature is that an end 202 of the existing plate 200 and the end cap 884A of the bands 872E align so that, for example, the same screw(s) or faster(s) may be used to affix both respective ends of the existing plate 200 and the band 872E by using holes 890A and 892A. Further, instead of a plate 200, the band assembly 870 of the present invention may be used on a level of vertebrae adjacent an artificial disc or other motion-preserving device (not shown). Similar to that shown in FIG. 25, in such an embodiment, the holes 890A and 892A of band 872E may align with affixation holes of the adjacent artificial disc or they may not.

Another benefit of the present invention is that the when placed on an anterior section of vertebral endplates, band 872 allows for a surgeon to stabilize that anterior portion of spine and subsequently, perform some work on the posterior side at or near the same section of vertebrae just stabilized. The flexibility of the band 872 will, for example, allow for a surgeon to work posteriorly with some degree of flexibility, while knowing that there is some degree of stabilization on the anterior side. Also, the band 872 allows a surgeon to turn a patient over, i.e., from the patient being on their backside (while working anteriorly) onto the patient's stomach with greater confidence than if the anterior portion was not stabilized prior to such a maneuver.

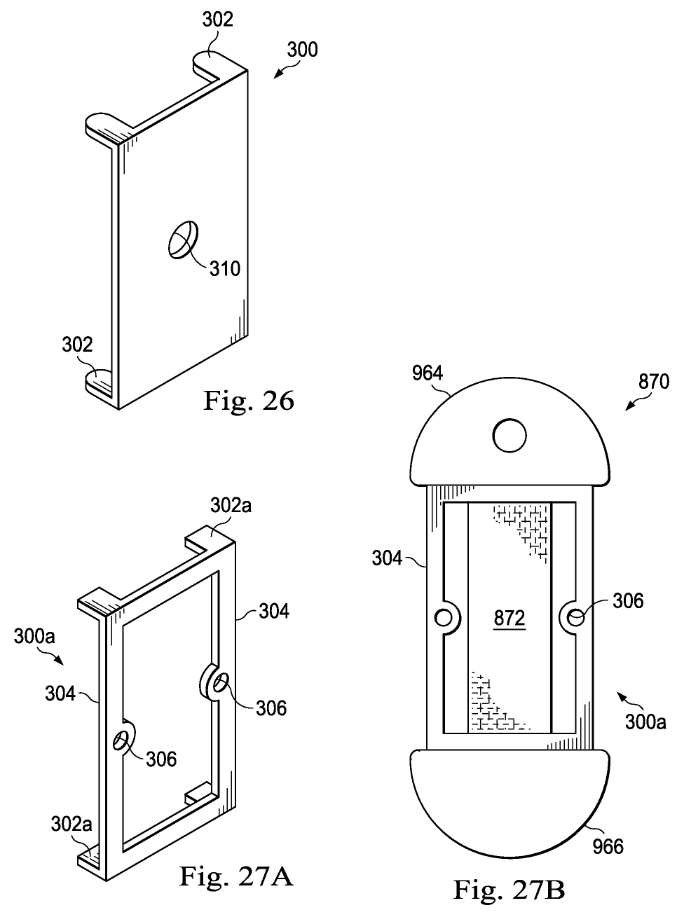

FIG. 26 shows a perspective schematic view of a holding device 300 for holding and inserting the band assembly 870 into position in the body, while FIG. 27A shows another perspective schematic view of another embodiment of such a device 300A. One way that the device 300 can be used is that after a band 872 is stretched to its desired tension, the top and bottom faces 302 of the holder 300 are positioned against the interior surfaces of the end caps 964, 966 of the band assembly 870, as shown in a front schematic view of FIG. 27B with respect to holder 300A. Holder 300A is used in the same manner, but also has distinct sides 304. After holder 300A is in position on a pre-tensioned band assembly 870 and attached or implanted in the proper location on the spine, for example, the sides 304 can be moved toward each other. This movement of the sides 304 toward each other will slightly reduce the length of the holder 300A, thereby releasing it from the tension band assembly 870 by removing the tension caused by the holder 300A itself. In the embodiment of 300A, holes 306 are provided on the sides 304 to facilitate the desired movement of the sides 304 toward each other, for example, with an instrument or instruments that can be inserted into holes 306. These holes 306 also will help with the act of holding and inserting of the band assembly 870.

Note that if a holder similar to holder 300 is used, hole 310 can be used to align the band assembly 870 with a spacer or graft over which the band 872 will be placed, e.g., if the spacer already has a corresponding hole in its center. For this purpose, hole 310 may alternatively be a relatively large hole or window for better visualization. In this way, the holder 300 can be used as an aid for proper placement of the assembly 870. Also, hole 310 also can help with the act of holding and inserting of the band assembly 870 with the aid of an appropriate instrument.

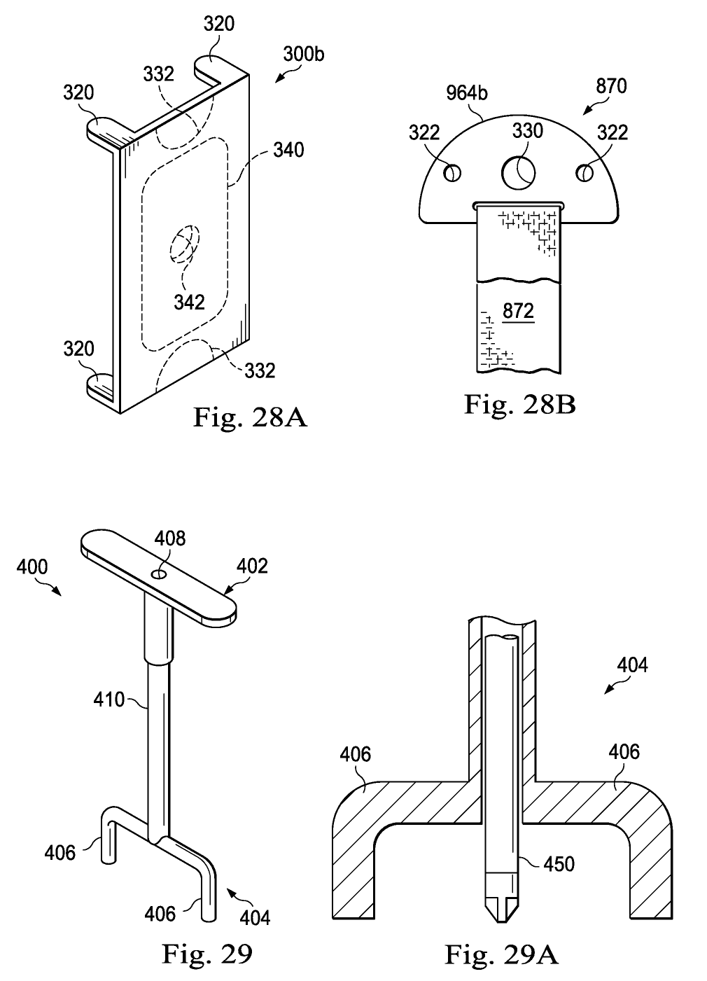

FIG. 28A shows an perspective schematic view of another embodiment of a holding device 300B, while FIG. 28B shows a cut-away schematic view of the embodiment of an end cap 964B of an embodiment of a tension band assembly 870 that works in cooperation with holder 300B. Holder 300B is used for the same purpose and similar manner as holder 300, but instead of the top and bottom faces 302 abutting the interior surfaces of the end caps 964, 966, holder 300B has four legs or posts 320 that cooperate with two holes 322 in each of the corresponding end caps of the band assembly 870. After the desired tension is achieved on band 872, posts 320 are positioned in each of holes 322 (two of which are shown in end cap 964B of FIG. 28B) to hold the band 872 in position. As with holder 300, holder 300B may have a window 340 or hole 342 in the plate that can be used as a visual indicator, e.g., used to align the band assembly 870 with a spacer or graft over which the band 872 will be placed, e.g., if the spacer already has a corresponding hole in its center. Further, holder 300B may have cut-outs 332 to provide ample space for a screw or fastener to be placed in connection hole 330 for attachment to a vertebra.

With respect to holders 300 and 300B, respectively, holes 310 and 342 may alternatively be replaced with a central post extending in the same direction as posts 320. In this way, the central post 310 or 342 may be used as an aid for proper placement of the assembly 870 by, e.g., using it to align with a spacer or graft over which the band 872 will be placed, e.g., if the spacer already has a corresponding hole in its center. Such an embodiment will advantageously have a corresponding hole in the band 872 at the location of the post 310 or 342. In addition, such a hole in the band 872 can be used to affix the band to the spacer or graft. Such a combination of a band 872 with a spacer or graft can be pre-assembled.

With the embodiments of holders 300, 300A, 300B, a band 872 of a band assembly 870 can be pre-tensioned and held in place with one of the holders at the latter stages of manufacturing so that the combination can be provided to a surgeon already pre-tensioned with the holder 300, 300A, 300B. Also, the holder 300, 300A, 300B can be color-coded to correspond to various sizes or tensions of bands 872 or band assemblies 870. Suitable materials for the holders 300, 300A, 300B can include, but not limited to, various metals, various polymers, and specific materials such as Celcon® and Delrin®.

When affixing an end cap 964, 882, 884 of the present invention in place on a vertebra or other location with, e.g., a screw, one challenge is preventing the end cap 964, 882, 884 from rotating. It is desirable to keep the end cap 964, 882, 884 in place so that the band 872 is in proper alignment and placement. One way to prevent this is by utilizing small protrusions such as spikes, teeth or pins on the underside of the end cap 964, 882, 884 so that it grabs into the surface of the vertebra. In addition to such anti-rotation devices, a guide may be used to hold the end cap 964, 882, 884 in place while inserting a screw through the end cap 964, 882, 884. FIG. 29 shows a schematic perspective view of an anti-rotation device 400 that can be used to address the problem of a rotating end cap 964, 882, 884 or assembly 870. As shown in FIG. 29, the anti-rotation device 400 comprises a proximal end 402, a hollow shaft 410 and a distal end 404 that has two feet 406 that extend away from the proximal end 402. FIG. 29A shows a cut-away, enlarged cross-sectional schematic view of the distal end of the anti-rotation device 400. In use, the proximal end 402 is designed to be hand-held by a user and the feet 406 are designed to cooperate with and be placed inside, e.g., holes 322 in an end cap 964B. When this occurs, the end cap 964 B is held in place by the user, and a screw driver 450 or other instrument for securing a fastener is inserted through a hole 408 in the anti-rotation device 400, thereby securing the end cap 964B to the vertebra in its desired position. The holes 322 may be in vertical alignment with connection hole 330, or may be offset, as shown, by example, in FIG. 28B. If offset, then the corresponding feet 406 on anti-rotation device 400 should be offset by the same amount, so that the feet 406, holes 322 and connection hole 330 all cooperate for proper functioning of the anti-rotation device 400.

All references cited herein are indicative of the level of skill in the art and are hereby incorporated by reference in their entirety.

The foregoing detailed description is to be clearly understood as being given by way of illustration and example only, the spirit and scope of the present invention being determined solely by the appended claims. For example, the band 872 or band assembly of the present invention in cooperation with anterior faces of vertebrae can alternatively be attached to other surfaces of vertebrae, e.g., lateral faces or lamina, or other parts of the body or spine, such as, e.g., facets, pedicles, pars, transverse processes or spinous processes. Note also while the present invention may be used in both any portion of the spine, including cervical and lumbar areas, when used in the lumbar region it may be advantageous to utilize a band 872 with greater stiffness than that utilized in the cervical section.

What is claimed is:

1. A device for linking first and second vertebral bodies having an implant therebetween, comprising:
a flexible member having first and second ends and being formed from a flexible material, said flexible member having a surface on which indicia is disposed for a visual and/or radiographic assessment of the tension in the flexible member; and
connector apparatus for securing the first and second ends of the flexible member to at least one of the first and second vertebral bodies.

2. The device of claim 1 wherein:
said flexible member is shaped in a loop.

3. The device of claim 2 wherein:
the surface on which indicia is disposed is a side surface of the tension member.

4. The device of claim 1 wherein:
said flexible member comprises a woven, knitted or braided fabric material.

5. The device of claim 1 wherein:
said flexible member comprises a treatment on at least a portion of the flexible member, said treatment adapted to promote bony integration between the flexible member and the first and second vertebral bodies to which it is connected.

6. The device of claim 1 wherein:
said flexible member comprises a treatment on at least a portion of the flexible member, said treatment adapted to reduce post-operative soft tissue adhesion to the flexible member.

7. The device of claim 1 wherein:
said flexible member comprises a therapeutic substance, and said flexible member further adapted to elute the therapeutic substance, over time, to a surgical site.

8. The device of claim 1 wherein the connector apparatus comprises:
first and second end caps, respectively secured to the first and second ends of the flexible member, said end caps comprising at least one opening formed therein through which fastening members may be extended to operatively secure the device to the first and second vertebral bodies.

9. The device of claim 1 wherein the connector apparatus comprises:
a plurality of mechanical fasteners extendable through the flexible member into at least one of the first and second vertebral bodies.

10. The device of claim 9 wherein:
the plurality of mechanical fasteners are selected from the group consisting of staples, screws, pins, tacks, expandable members and fasteners formed from a shape memory material.

11. The device of claim 1 wherein:
tunnels extend into at least one of the first and second vertebral bodies,
portions of the flexible member are positionable in the tunnels, and
the connector apparatus comprises a plurality of fastening members, formed from shape memory material or having radially expandable structures, positionable within the tunnels to bear against the flexible member portions and captively retain them in the tunnels.

12. The device of claim 1 wherein the connector apparatus comprises:
a plurality of connector structures, connectable to the first and second ends of the flexible member, for securing the first and second ends of the flexible member to at least one of the first and second vertebral to provide an obstacle at least in part to the dislodgement of the implant from between them, and adapted to apply tension to the vertebral bodies, each of the connector structures comprising:
a hollow locking structure with a base wall having an opening therein and being securable to one of the first and second ends of the flexible member, and an outer wall spaced apart from and parallel to the base wall, the outer wall having an opening therein which opposes the base wall opening and is partially bounded by one or more resiliently deflectable lobe portions of the outer wall, and
a screw member extendable sequentially through the outer wall opening, the base wall opening and the one of the first and second ends of the flexible member, and threadable into one of the first and second vertebral bodies, the screw member further having a transversely enlarged head portion configured to resiliently deflect the one or more lobe portions, as the screw member passes through the interior of the hollow locking structure, and then permit the one or more deflected lobe portions to snap back to their undeflected positions in which they block outward passage of the head portion through the outer wall opening.

13. A device for linking first and second vertebral bodies having an implant therebetween, comprising:
a linking member having first and second ends; and
a plurality of connector structures, each connectable to one of the first and second ends of the linking member, for securing the first and second ends of the linking member to at least one of the first and second vertebral bodies and adapted to provide an obstacle to the dislodgement of the implant from between them, wherein:
each of the first and second ends of the linking member comprising a mounting hole extending therethrough, and
the plurality of connector structures comprise:
first and second screw members each having a head portion through which an opening extends axially inwardly, the first and second screw members being respectively threadable into the first and second vertebral bodies,
first and second elongated guide members, each configured to be respectively and removably inserted into the openings in the first and second screw members, after the screw members are threaded into their associated vertebral bodies, and to thereafter have longitudinal portions projecting outwardly from the first and second screw members, the longitudinal portions of the guide members being movable away from one another upon application of a separation force thereon to increase the separation distance between the first and second vertebral bodies, and configured such that outwardly projecting portions extend through the mounting holes of the linking member, and
first and second locking members constructed and operative to respectively lock the first and second linking member ends to the head portions of the first and second screw members after the first and second elongated guide members are respectively removed from the first and second screw member openings.

14. The device of claim 13 wherein:
the linking member is constructed from a flexible material.

15. The device of claim 14 wherein:
the linking member is an elastic tension band.

16. The device of claim 13 wherein:
the linking member is a rigid member.

17. Apparatus for linking first and second vertebral bodies having a third vertebral body disposed therebetween, comprising:
a flexible structure having first and second ends, and an elongated slot extending through a longitudinally intermediate portion of the flexible structure and longitudinally extending parallel to the length of the flexible structure;
first connection structure for respectively securing the first and second ends of the flexible structure to the first and second vertebral bodies; and
second connection structure, extendable through the slot through a selectively variable longitudinal portion thereof and securable to the third vertebral body.

18. The apparatus of claim 17 wherein:
said flexible structure comprises an elastic material.

19. The apparatus of claim 17 wherein:
said flexible structure comprises approximately a looped configuration, and the first connection structure comprises first and second end caps having slots therein through which the first and second end portions of the flexible structure respectively extend, and openings through which fasteners may be extended and positioned into the first and second vertebral bodies.

20. The apparatus of claim 17 wherein:
the second connection structure comprises a grommet configured to outwardly overlie the flexible structure at a portion of the slot, and a screw extendable through the grommet and threadable into the third vertebral body.
